(19) United States
(12) Patent Application Publication
(10) Pub. No.: US 2026/0335017 A1
(43) Pub. Date: Oct. 29, 2026

(54) CUT-PATH VISUALIZATION FOR ACTIVE SAW CUTTING

(71) Applicant: Zimmer, Inc., Warsaw, IN (US)

(72) Inventor: Kamran Shamaei, Austin, TX (US)

(21) Appl. No.: 19/647,383

(22) Filed: Apr. 14, 2026

Related U.S. Application Data
(60) Provisional application No. 63/788,872, filed on Apr. 15, 2025.

(51) Int. Cl. A61B 34/10; A61B 17/14; A61B 34/32; A61B 90/00

(52) U.S. Cl. CPC A61B 34/10; A61B 17/142; A61B 34/32; A61B 90/37; A61B 2034/105; A61B 2034/107; A61B 2034/108

(57) ABSTRACT

A technique for generating a user interface (UI) that displays planned, in-process, and past cut paths for an autonomous surgical robot performing a surgical procedure on patient anatomy is described. The UI concurrently presents one or more graphical representations of already-performed cut paths, a currently active cut path, and/or one or more to-be-performed cut paths, each rendered with visually distinguishable encodings to convey temporal state. The technique includes identifying one or more planned cut paths that an autonomous robotic surgical system is to follow in cutting patient anatomy, where the cutting tool can include a blade that moves in an oscillating or reciprocating motion. The UI includes virtual representations of the patient anatomy and the identified planned cut paths, including positioning and trajectory thereof relative to the patient anatomy. Virtual boundary constraints corresponding to at-risk anatomic structures may be displayed spatially relative to the cut paths.

### CLAIM OF PRIORITY

This application claims the benefit of U.S. Provisional Patent Application Ser. No. 63/788,872, filed on Apr. 15, 2025, the benefit of priority of which is claimed hereby, and which is incorporated by reference herein in its entirety.

### BACKGROUND

Total Knee Arthroplasty (“TKA”), commonly referred to as a “knee replacement”, is a procedure of orthopedic surgery in which a knee joint, such as an arthritic knee joint, is replaced with a prosthesis. In a knee replacement, a series of bone resections are made to accommodate the placement of implants.

### SUMMARY

Shortcomings of the prior art are overcome and additional advantages are provided through the provision of method(s) described herein, and computer system(s) and program product(s) configured to perform such method(s) described herein.

More specifically, user interfaces for displaying cut paths of an active/autonomous robot performing a TKA or similar orthopedic surgical procedure are described. The user interfaces can display currently active cut paths as well as past and future cut paths followed or to be performed by the robotic system. In some examples discussed herein, the autonomous robot utilizes a sagittal saw to perform pre-programmed resections of a bone as part of an orthopedic surgical procedure, such as a TKA. As discussed herein, the autonomous robot can utilize multiple different cut paths for perform any particular resection to avoid soft tissue or to minimize cutting time required for the resection. In aspects described herein, user interface concepts provide visualization of cut paths, among other features. In some examples, the visualization discusses are particularly tailored to provide a surgeon operating the robotic platform with feedback as to how the autonomous robotic platform will perform certain resections. The user interfaces can also assist in visualizing how past resections were performed, which can be particularly useful in understanding how the autonomous robotic platform adapted certain cut paths during the resection procedure.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Other aspects and features will become apparent from the following detailed description considered in conjunction with the drawings.

### BRIEF DESCRIPTION OF THE DRAWINGS

Aspects described herein are particularly pointed out and distinctly claimed as examples in any claims at the conclusion of the specification. The foregoing and other objects, features, and advantages of the disclosure are apparent from the following detailed description taken in conjunction with the accompanying drawings in which:

FIG. 1 depicts example resections of a total knee arthroplasty;

FIG. 2 illustrates oscillatory motion of a sagittal cutting blade;

FIG. 3 illustrates an example of displayed cut path(s) for a rotary tool;

FIG. 4 depicts an example of bone cutting using a haptic robotic surgical system;

FIGS. 5 and 6 depict examples of sagittal cut path display, in accordance with aspects described herein;

FIGS. 7A-12B depict examples of anatomic structures for avoidance and corresponding cut path visualization in accordance with aspects described herein for various cuts of a TKA procedure;

FIG. 13 depicts an example process for cut path visualization in accordance with aspects described herein; and

FIG. 14 depicts an example computer system to incorporate, use, and/or facilitate aspects described herein.

### DETAILED DESCRIPTION

Described herein are aspects (e.g., methods, systems, computer program products) to facilitate robotic surgical procedures, including display of cut paths for an active/autonomous robot on a user interface, and display of a cutting tool's progress on each such cut path. Cut paths for cuts that were already-performed, are in the process of being performed, and/or are to-be-performed may be displayed, in examples. Aspects may be particularly helpful for robotic and non-robotic navigated surgical procedures, and could have additional/other industrial applications.

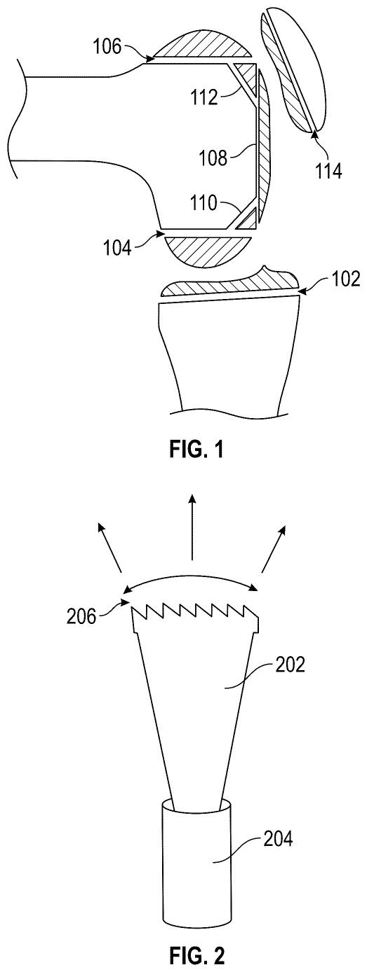

Examples of aspects provided herein are described with reference to a TKA by way of example only, and not limitation. FIG. 1 depicts example cuts of a TKA, which generally includes seven planar cuts: five femoral cuts to the patient's femur, one tibial cut to the patient's tibia, and one patella cut to the patient's patella. These can all be achieved with a cutting instrument, for example a sagittal saw. The seven planar resections are identified in FIG. 1 as a tibial resection 102, a posterior femur resection 104, an anterior femur resection 106, a distal femur resection 108, two chamfer resections (a posterior chamfer resection 110 that provides a chamfer between the posterior femur resection and the distal femur resection, and an anterior chamfer resection 112 that provides a chamfer between the anterior femur resection and the distal femur resection), and a patella resection 114.

All of the cuts can be executed with a sagittal saw, as an example. A sagittal saw includes (at least) a planer saw blade and a drive mechanism. The planar saw blade extends distally from the drive mechanism, which moves the saw blade in an oscillatory, back-and-forth pattern (a cutting stroke) in the plane in which the blade is aligned, and generally in a motion/arc that is oriented perpendicular to the direction in which the saw is advanced forward to achieve the desired cut. In a traditional TKA procedure, a surgeon will typically utilize one or more cut guides to guide the sagittal saw to perform the series of cuts (resections) described in reference to FIG. 1. The autonomous robotic platform described herein can perform the resections for a TKA without use of an external cut guide, which allows for certain tool paths or cut paths that may be common in a traditional TKA procedure. Further, the autonomous robotic platform may adapt a pre-programmed cut path during a resection to automatically compensate for bone density changes, soft tissue issues, or other anatomic deviations not anticipated in the planning process.

FIG. 2 illustrates oscillatory motion of a sagittal cutting blade, specifically depicting an example sagittal cutting blade 202 that oscillates (left and right in this figure) and is coupled to/mounted in a base portion 204. The cutting blade has teeth 206 that cut only when the teeth, oscillating as the blade oscillates, move across the target material. The cutting tool, and more specifically the blade thereof when oscillating, is driven forward (indicated by the arrows in FIG. 2) into the material to be cut.

Robot-assisted surgical procedures are becoming increasingly popular. This includes TKA surgeries performed with the use of a robot/robotic surgical system. An active/fully autonomous robot is able to perform cuts without requiring direct force to be applied to the arm or cutting tool by the surgeon. Generally, there is some level of pre-programmed instruction(s) and real-time feedback to execute bone resections with minimal or no surgeon intervention. In contrast, haptic-based robots require a user to apply direct force to the arm or cutting tool. In haptic-based systems, the surgeon's hand guides the tool within robot-enforced boundaries (e.g., with haptic feedback) to prevent the surgeon from cutting outside of the planned area. A key difference between an active robot and a haptic-based robot is that active robots work autonomously with surgeon oversight, while haptic robots assist the surgeon without taking full control.

Conventional active/autonomous robotic surgical systems cut using a rotary tool, and any programmed cut paths displayed for viewing correspond to tool paths executed with a rotary tool by the active robot. More recently, active/autonomous robots have been developed that utilize a robot-mounted saw (e.g., a sagittal saw) as the cutting tool. Accordingly, the cut path displays discussed herein are focused on displaying aspects of the cut/tool paths unique to sagittal saw cutting with an autonomous robot and could also be applicable to a navigated non-robotic sagittal saw.

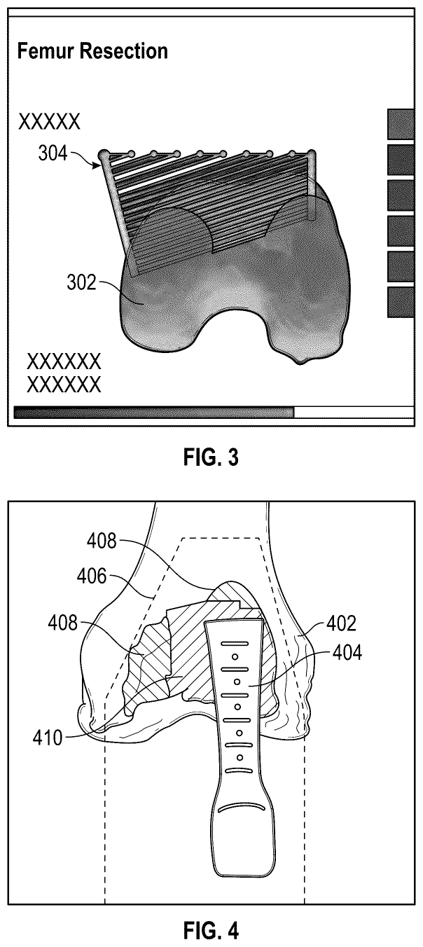

FIG. 3 illustrates an example of displayed cut path(s) for a rotary tool, in which a cut being performed on patient bone 302 is defined by cut path(s) 304, which is/are a sequence of linear sweeps in a direction perpendicular to the direction of the cut to be made. The sequence of linear sweeps have steps between them to create a zig-zag pattern, forming a cut plane that enters and continues through the patient bone to accomplish the resection.

The cut path(s) of a rotary cutting tool are significantly different from cut paths of sagittal saw for cutting by an active robotic system. Haptic robotic systems, which also common utilize a rotary cutting tool, typically leave the user unconstrained in terms of in-plane cutting-the user decides the cut paths often while or as the user performs the cutting. The user is free to move the cutting tool within a constrained plane but established virtual boundaries prevent the cutting blade from straying undesirably far from the intended resection area. In these situations, no cut paths are displayed.

FIG. 4 is an in-process autonomous robotic resection to a patient bone 402 utilizing a sagittal saw. A top view is shown in which the saw blade 404 is in the process of performing the resection (for instance to perform an anterior femoral resection of a TKA). Here, a virtual boundary 406 serves to illustrate the cut plane or virtual boundary that would constrain the autonomous robot from moving the saw blade outside of the boundary, for instance to protect surrounding soft tissue, as an example. The areas 408 denote virtually-imposed, in-plane areas where the user has not yet cut. Area 410 denotes the in-plane area where the saw has cut.

A guidance application for a navigated TKA system is a software-driven interface that assists surgeons by providing real-time visual feedback during the surgical procedure, for instance during cutting. For navigated robotic TKA applications, it may be beneficial to the surgeon for the guidance application to display cut paths. Conventional guidance applications for navigated TKA systems do not display cut paths for an active/autonomous robot utilizing a robot mounted saw (e.g., sagittal saw) to accomplish cut(s), for instance cuts to bone.

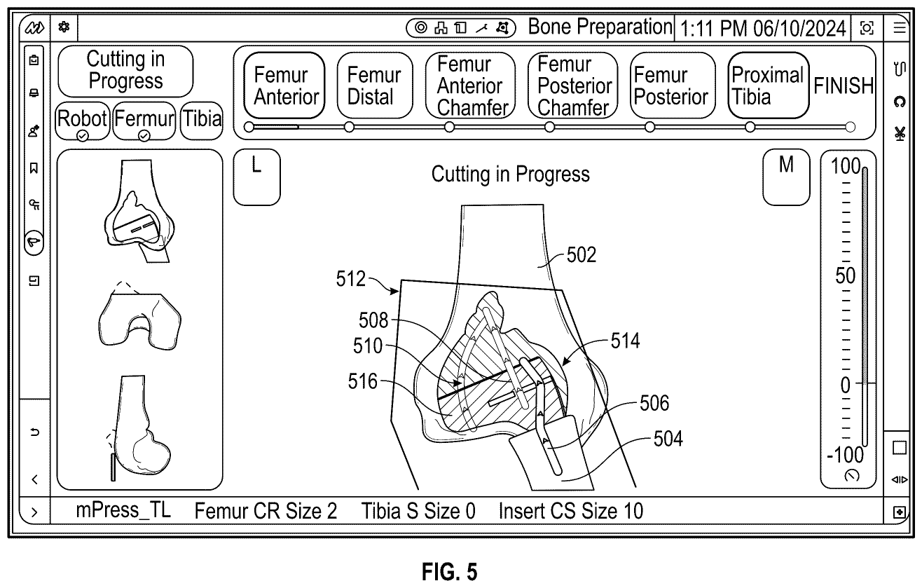

FIG. 5 depicts example sagittal cut paths displayed in accordance with aspects described herein to show the user (e.g., a surgeon) where the cutting tool will advance or retract, in accordance with aspects described herein. In embodiments, each cut path is a collection of waypoints that the autonomous robot cutting tool is to follow during a cutting operation. Shown in this example are cut paths for an anterior femur cut to patient bone 502. Specifically, for sagittal saw cutting blade 504, three cut paths—506, 508, and 510—are shown. Paths 506 and 510 show curved cut paths that the blade trajectory is to follow. In examples, the real-time tool position may be overlaid on the path, and this may be done by imposing graphical element(s) representing the blade. Various coloring, shading, or other visual distinctions can be imparted to the respective cut paths and/or the particular bone anatomy to be encompassed by the respective paths to show an order or relation as between two or more paths. For instance, the system could show a current cut path (say 506, corresponding to a cut in progress) in a first color and/or shading, and a next cut path (say 508 or 510 corresponding to a next cut to perform) in a different color and/or shading. Any cut paths for cuts performed after that next cut could be in yet a third color and/or shading. The user interface 500 also includes illustration of virtual boundary 512 (also referenced as cut plane 512), cut area 516 and uncut area 514.

To perform a function like cutting, an active robot is to move through a series of ordered coordinates, collectedly referred to as a toolpath, which, in the case of a cutting blade, is a cut path. Generally, the tool executes its intended task at or around each ordered coordinate on the toolpath. For TKA applications, an active/autonomous robots can utilize a saw (e.g., sagittal saw), and display toolpaths for a sagittal saw. Display of these toolpaths on a guidance application can inform the user of the real-time position of the cutting tool relative to a current tool path (i.e., cut path of the in-progress cut), a future tool path (i.e., a cut path of a to-be-performed cut) and/or a past tool path (i.e., a cut path of an already-performed cut) to provide visual feedback on how the robot utilized the saw during a TKA.

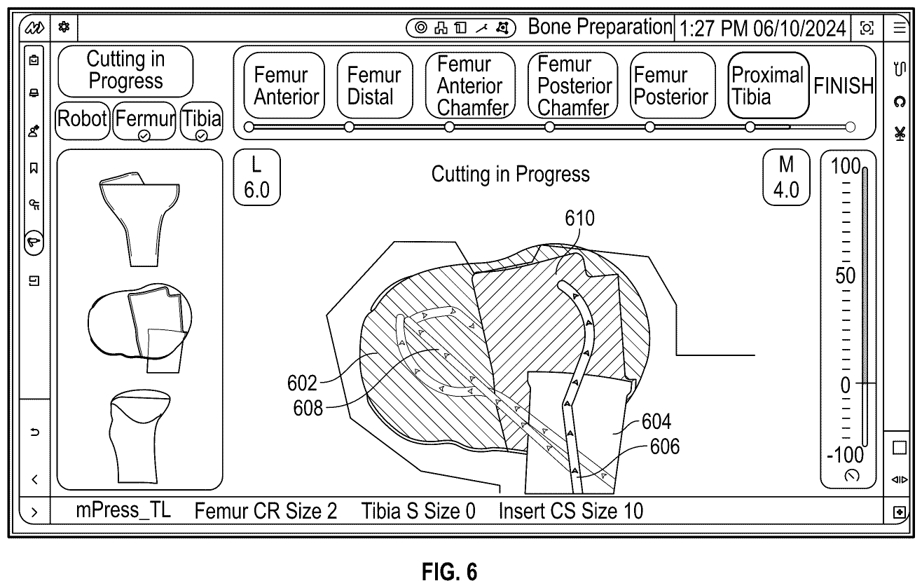

FIG. 6 depicts another example of a display of sagittal cut paths in accordance with aspects described herein. Shown in this example are cut paths for a proximal tibial cut to patient bone 602. For saw blade 604, shown are a current cut path 606 of an in-process cut and a next cut path 608 for a next cut. Subsequent cut paths are also shown. FIG. 6 also includes various other cut paths the device may perform that are not referenced with reference numbers.

Without displayed cut paths, the user would have limited information regarding positions of the cutting tool during past (performed), present (in-process), and future (to-be-performed) cutting. Cut path visualization is important for various reasons that include, but are not limited to:

Surgeon Oversight and Trust—Cut path visualization makes it easy to validate the accuracy of the robot in terms of execution of the cuts, for instance that the robot has, is, and/or will follow the cut path(s) that is generated. Additionally, it is much more difficult to validate that a unique cut path (generated in the surgery and generally not modifiable absent special circumstances) is perfect, optimal, or the like. By visualizing the cut paths, the surgeon can verify that the cut path is safe and reasonable, and in the worst case avoid it altogether. Additionally, sometimes there is severe deformity, osteophytes, unusual anatomy, etc. Displaying the cut paths can be an important safety check that the robotic plan is going to work well.

Inform surgeon when the cuts are dangerous—Without displayed cut paths, the surgeon would be uninformed about where the robot is to cut next. It could be imperative to know when cut paths are going to be close to critical soft tissue structures to plan accordingly (for example by repositioning the knee and/or placing retractors). By way of example, consider cutting without a retractor on the patellar tendon where the patellar tendon is not dislocated. If the cut path is not visualized, the surgeon may not appreciate that extreme caution is needed to be exercised related to that tendon in particular. Additionally, most surgeons would desire that a retractor be used in this situation, cut path visualization can provide an opportunity to insert retractors or intervene where an anomaly may suggest it is warranted.

Intraoperative Communication—Individuals present in the operating room each have respective roles. For example, an assistant might need to hold retractors or clear anatomical debris such as bone chips. The robot operator may need to engage the safety stop or similar safety provision. Displaying cut paths, particularly for visualization on a display that each of the individuals can view enable them all to know exactly what is happening in terms of cutting, which can be advantageous particularly given the loudness of the cutting tool and the potential difficulty of verbal communication in that situation.

Data analysis and optimization—Displayed cut paths can be analyzed post-operatively for continuous improvement, for instance by associating post-operative outcomes with the employed cut paths to identify both cut paths producing desirable outcomes and cut paths producing undesirable outcomes.

Recutting—Visualizing the cuts allows the surgeons to initiate recutting a given cut, which might be desired in some situations. If the cut was not visualized, the surgeon may not know which cut needed is to be re-executed.

Thus, aspects described herein propose displaying cut paths on a user interface (e.g., in a guidance application) for an active/autonomous robot utilizing a robot mounted saw based cutting tool. Specifically disclosed, in embodiments, is the display of a series of cut paths that help facilitate robotic execution of TKA cuts with a sagittal cutter mounted to an active robot. Conventional active robotic surgical systems that execute TKA bone cuts do not do so with a saw (e.g., sagittal saw), and therefore to not display saw cut paths for performed, in-process and/or to-be-performed cuts.

Advantages of cutting with a sagittal saw rather than a rotary tool include speed of cutting and mitigating the risks of thermal necrosis. The advantages of an active robot verses a haptic robot may include, but are not limited to: speed, increased freedom for the surgeon to focus on other surgical tasks (for example having two hands to manipulate retractors), better execution of challenging cuts (for example milling a hip cavity), and generally less demands on the user. Aspects described herein can display cut paths for an active robot using a sagittal saw on a user interface and show the progress of the cutting tool on these respective cut paths.

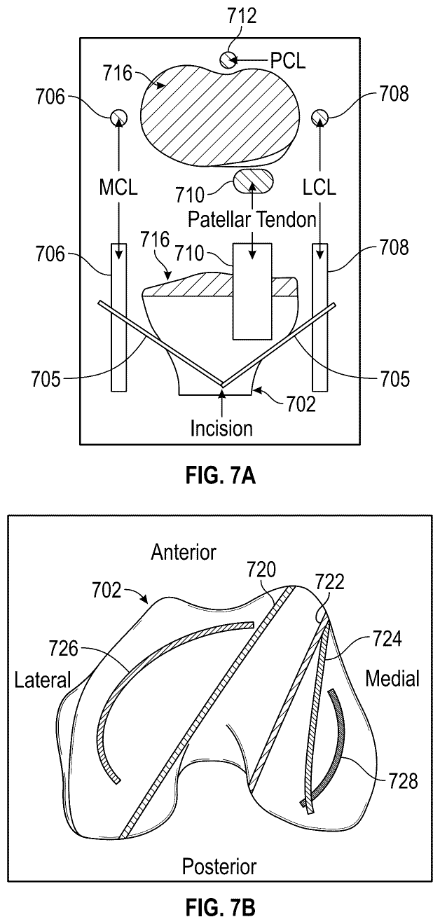

Further details are now provided using the examples of FIGS. 7A-12B, which depict examples of anatomic structures for avoidance and corresponding cut path visualization in accordance with aspects described herein for various cuts of a TKA procedure (all of those noted above except the patella cut).

In general, FIGS. 7A, 8A, 9A, 10A, 11A and 12A depict access to the surgical site for a given resection and the sensitive anatomical structures at risk during the cut for the example TKA procedure. A system may display gates (virtual constraints) which simulate the lateral constraints of a virtual jig designed to constrict the trajectory and motion of the blade relative to sensitive tissue structures to protect them. These gates can be visualized on the display/interface/screen along with the cut paths. In general, FIGS. 7B, 8B, 9B, 10B, 11B, and 12B depict visualizations of the cut paths themselves.

FIG. 7A conceptually illustrates clinical constraints with respect to cutting performed for the proximal tibia cut. FIG. 7B depicts corresponding proposed cut paths, such as cut paths 720, 722, 724, 726, and 728, which are built based on a preoperative plan and conveyed in a graphical interface for a user. Referring to FIG. 7A, the tibia 702 is illustrated with a typical incision 705 and the primary critical structures at risk during the proximal tibial resection that include the patellar tendon, medial collateral ligament (MCL) 706, lateral collateral ligament (LCL) 708, patellar tendon 710, and posterior cruciate ligament (PCL) 712. An example optimal entry is highlighted in FIG. 7B by 703, with trajectories of cut paths originating in the anterior medial bone region between the MCL 706 and patellar tendon 710. As shown, cut path. 726 includes a counterclockwise trajectory (anterior to posterior) along the lateral bone boundary (shown as trajectory labeled 3) and cut path 728 includes a clockwise trajectory (anterior to posterior) along the medial bone boundary. Cut paths 720, 722, and 724, by way of non-limiting example, remove the remaining bone. FIG. 7A also includes illustration of the cut areas 716 on the proximal tibia, which represent the target bone to be resected using cut paths 720, 722, 724, 726, and 728 in this example.

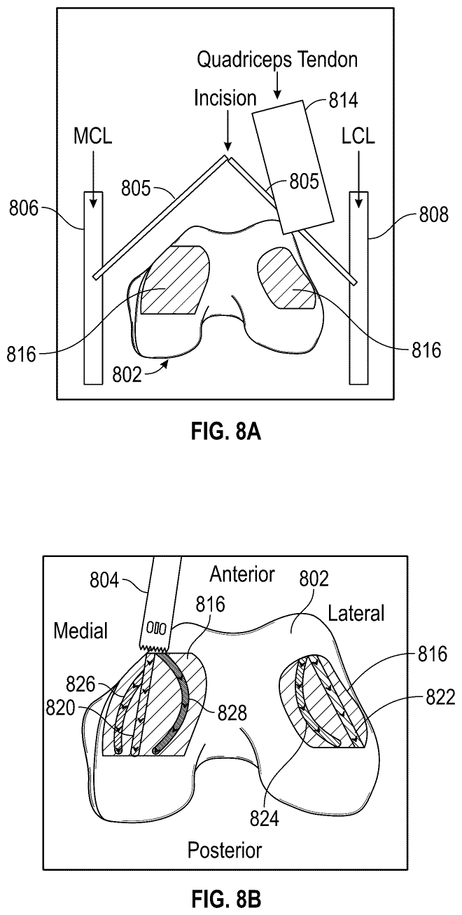

FIG. 8A conceptually illustrates clinical constraints with respect to cutting performed for the distal femur cut. FIG. 8B depicts corresponding proposed cut paths, such as cut paths 820, 822, 824, 826, and 828, which are built based on a preoperative plan and conveyed in a graphical interface for a user. Referring to FIG. 8A, this distal end of a femur 802 is illustrated along with an incision 805 and the primary critical structures at risk during the distal femur resection include the Quadriceps Tendon 814, MCL 806, and LCL 808. An example optimal entry is shown in FIG. 8B by a position of blade 804 at the anterior side. Trajectories of cut paths are shown by numbered paths/trajectories, cut paths 820, 822, 824, 826, and 828. In this example, the blade 804 cuts generally anterior to posterior along each of the cut paths 820, 822, 824, 826, and 828. On the medial condyle, the blade 804 follows a counterclockwise trajectory (anterior to posterior) along the medial bone boundary (shown as cut path 826). On the lateral side of the medial condyle, the blade 804 cuts in a clockwise trajectory (anterior to posterior) along the lateral bone boundary (shown as cut path 822). On the lateral condyle, the blade 804 cuts in a counterclockwise trajectory along the medial bone boundary (shown as cut path 824) and follows a generally clockwise trajectory on the lateral condyle along the lateral bone boundary (shown as cut path 828). FIGS. 8A and 8B also include illustration of the cut areas 816 on the femoral condyles, which represent the target bone to be resected using cut paths 820, 822, 824, 826, and 828.

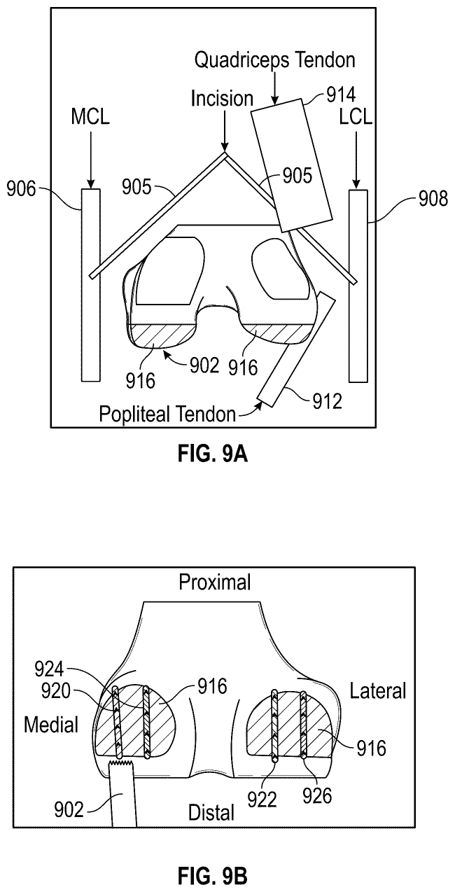

FIG. 9A conceptually illustrates clinical constraints with respect to cutting performed for the posterior femur cut. FIG. 9B depicts corresponding proposed cut paths which could be built and conveyed in a graphical interface for a user. Referring to FIG. 9A, the primary critical structures at risk during the posterior femur resection include the Quadriceps Tendon 914, MCL 906, popliteal tendon 912, and LCL 908. An example optimal entry is shown in FIG. 9B by a position of blade 902 at the distal side. Trajectories of cut paths are shown by numbered paths/trajectories 920, 922, 924, and 926. The blade 902 cuts distal to proximal. On the medial condyle, the blade 902 follows a clockwise linear trajectory (distal to proximal) along the medial bone boundary (shown as trajectory labeled 920). On the lateral side of the medial condyle, the blade 902 cuts in a counterclockwise linear trajectory (distal to proximal) along the lateral bone boundary (shown as trajectory labeled 924). On the lateral condyle, the blade 902 cuts in a clockwise trajectory along the medial bone boundary (shown as trajectory labeled 922) and follows a generally counterclockwise linear trajectory on the lateral condyle along the lateral bone boundary (shown as trajectory labeled 926). FIGS. 9A and 9B also include illustration of the cut areas 916 on the posterior femoral condyles, which represent the target bone to be resected using cut paths 920, 922, 924, and 926.

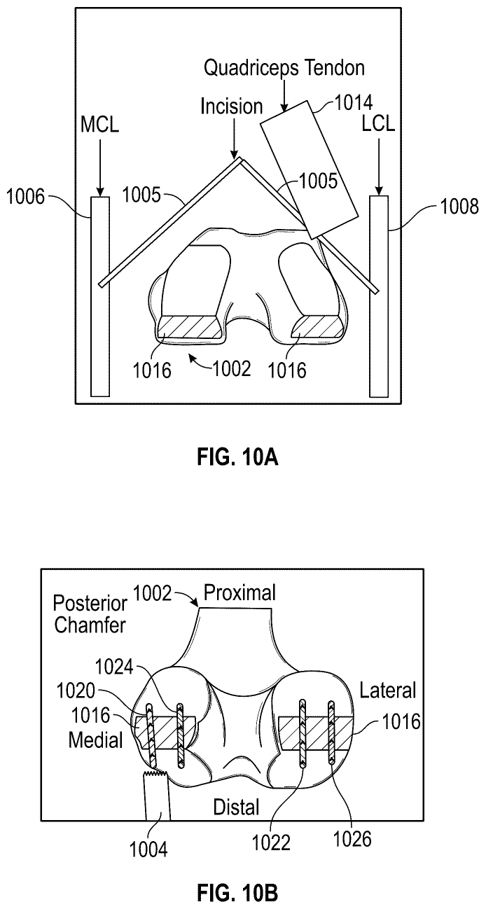

FIG. 10A conceptually illustrates clinical constraints with respect to cutting performed for the posterior chamfer cut. FIG. 10B depicts corresponding proposed cut paths, such as cut paths 1020, 1022, 1024, and 1026, which are built based on a pre-operative plan and conveyed in a graphical interface for a user. Referring to FIG. 10A, the distal femur 1002, incision 1005, and the primary critical structures at risk during the posterior femur resection include the Quadriceps Tendon 1014, MCL 1006, and LCL 1008. An example optimal entry is shown in FIG. 10B by a position of blade 1004 at the distal end on the posterior side. Trajectories of cut paths are shown by numbered cut paths 1020, 1022, 1024, and 1026. The blade 1004 cuts posterior to anterior on a distal end of the femur. On the medial condyle, the blade 1004 follows a linear trajectory (posterior to anterior) along the medial bone boundary (shown as cut path 1020). On the lateral side of the medial condyle, the blade 1004 cuts in a linear trajectory (posterior to anterior) along the lateral bone boundary (shown as cut path 1024). On the lateral condyle, the blade 1004 cuts in a linear trajectory along the medial bone boundary (shown as cut path 1022), and follows a generally linear trajectory on the lateral condyle along the lateral bone boundary (shown as cut path 1026). FIGS. 10A and 10B also include illustration of the cut areas 1016 on the distal femur, which represent the target bone to be resected using cut paths 1020, 1022, 1024, and 1026.

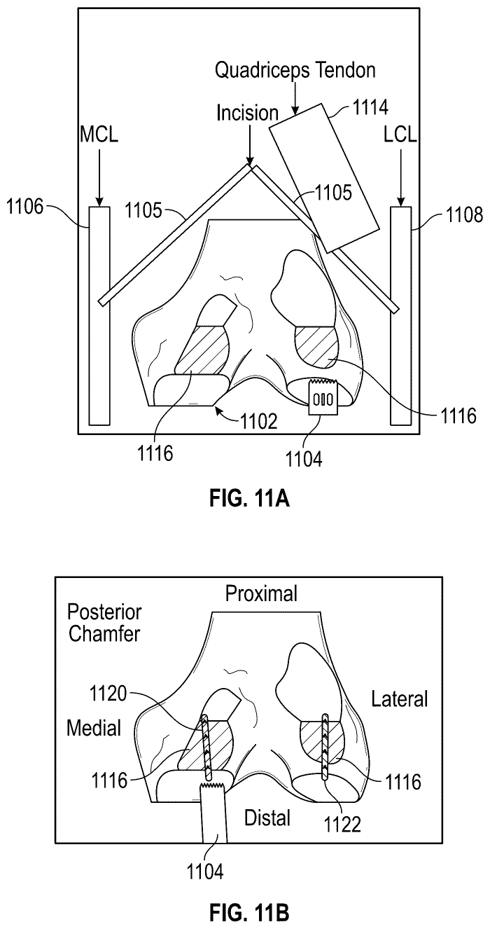

FIG. 11A conceptually illustrates clinical constraints with respect to cutting performed for the anterior chamfer cut, depicted as cut areas 1116. FIG. 11B depicts corresponding proposed cut paths 1120 and 1122 which are generated based at least in part on a pre-operative plan and conveyed in a graphical interface for a user. Referring to FIG. 11A, the anterior side of the distal femur 1102, incision 1105, and primary critical structures at risk during the anterior chamfer cut include the Quadriceps Tendon 1114, MCL 1106, and LCL 1108. An example optimal entry is shown in FIG. 11B by a position of blade 1104 at the distal side. Trajectories of cut paths are shown by numbered paths/trajectories 1 and 2. The blade 1104 cuts distal to proximal. On the medial condyle, the blade 1104 can generally execute the cut in a single linear distal to proximal trajectory (shown as trajectory labeled 1). On the lateral condyle, the blade 1104 generally cuts in a single linear distal to proximal trajectory (shown as trajectory labeled 2). FIGS. 11A and 11B include illustration of the cut areas 1016 on the distal anterior of the femur, which represent the target bone to be resected using cut paths 1120 and 1122.

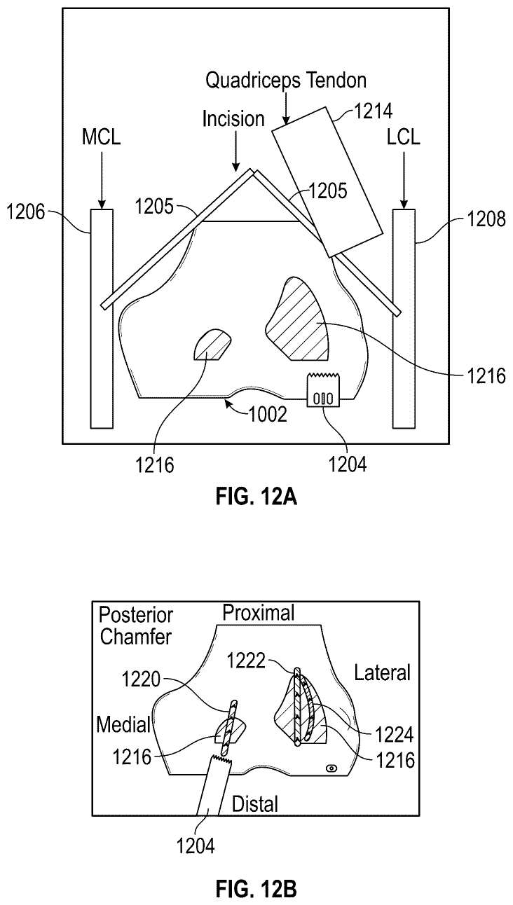

FIG. 12A conceptually illustrates clinical constraints with respect to cutting performed for the anterior femur cut, depicted as cut areas 1216. FIG. 12B depicts corresponding proposed cut paths 1220, 1222, and 1224 which are generated based on a pre-operative plan and conveyed in a graphical interface for a user. Referring to FIG. 12A, the anterior side of the distal femur 1202, incision 1205 and the primary critical structures at risk during the anterior femur cut include the Quadriceps Tendon 1214, MCL 1206, and LCL 1208. An example optimal entry is shown in FIG. 12B by a position of blade 1204 at the distal side. Trajectories of cut paths are shown by numbered cut paths 1220, 1222, and 1224. The blade 1104 cuts distal to proximal. On the medial condyle, the blade 1204 follows a generally linear distal to proximal trajectory (shown as cut path 1220). On the medial side of the lateral condyle, the blade 1204 cuts in linear trajectory (distal to proximal) along the medial bone boundary (shown as cut path 1222). On the lateral side of the lateral condyle, the blade 1204 cuts in a counterclockwise trajectory along the lateral bone boundary (shown as cut path 1224).

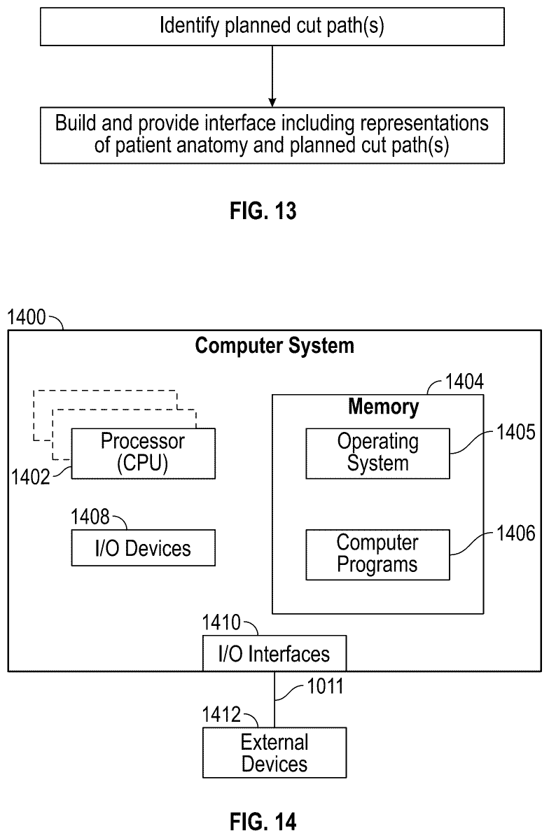

Accordingly, FIG. 13 depicts an example process for cut path visualization. The process may be executed, in one or more examples, by a processor or processing circuitry of one or more computers/computer systems, such as those described herein. In one example, program code or instructions may, based on execution thereof by a processing circuit/processor(s), implement one, some, or all aspects of FIG. 13.

Referring to FIG. 13, the process includes identifying, as part of a surgical plan, one or more planned cut paths that a robotic surgical system is to follow in cutting patient anatomy using a saw as part of a surgical procedure. each cut path of the one or more planned cut paths defines a respective path along which the robotic surgical system is to drive a blade of the saw in performing a respective cut. The process also includes building and providing (displaying, presenting, conveying) an interface. The building includes in the interface: virtual representations of the patient anatomy; and the identified one or more planned cut paths (i.e., graphical representations of the cut path(s)), including positioning and trajectory thereof relative to the patient anatomy.

Aspects described herein can be helpful for surgical navigation technology/systems, navigated surgical procedures, and other industrial applications. Aspects could be integrated into robotic surgical systems, for example. In some embodiments, aspects are provided as software that can be integrated into target systems. There may be applications outside of surgery.

One or more embodiments described herein may be incorporated in, performed by, and/or used by one or more computer systems, such as one or more systems that are, or are in communication with, a camera system, tracking system, and/or orthopedic surgical robot, as examples. Processes described herein may be performed singly or collectively by one or more computer systems. A computer system may also be referred to herein as a data processing device/system, computing device/system/node, or simply a computer. The computer system may be based on one or more of various system architectures and/or instruction set architectures.

FIG. 14 depicts an example computer system to incorporate, use, and/or facilitate aspects described herein. Computer system 1400 may be provided as part of a surgical navigation technology/system, for example. Computer system 1400 is in communication with one or more external device(s) 1402 (such as one or multiple robot(s), tracking camera(s), rigid robot tracking array(s), foot pedal(s), monitor(s), Deadman switch(es), etc.). Computer system 1400 includes one or more processor(s) 1402, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor of processor(s) 1402 can also include register(s) to be used by one or more of the functional components. Computer system 1400 also includes memory 1404, input/output (I/O) devices 1408, and I/O interfaces 1410, which may be coupled to the processor(s) 1402 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhances ISA (EISA), the Video Electronics Standards Association (VESA) local bus, and the Peripheral Component Interconnect (PCI).

Memory 1404 can be or include main or system memory (e.g., Random Access Memory) used in the execution of program instructions, storage device(s) such as hard drive(s), flash media, or optical media as examples, and/or cache memory, as examples. Memory 1404 can include, for instance, a cache, such as a shared cache, which may be coupled to local caches (examples include L1 cache, L2 cache, etc.) of processor(s) 1402. Additionally, memory 1404 may be or include at least one computer program product having a set (e.g., at least one) of program modules, instructions, code or the like that is/are configured to carry out functions of embodiments described herein when executed by one or more processors.

Memory 1404 can store an operating system 1405 and other computer programs 1406, such as one or more computer programs/applications that execute to perform aspects described herein. Specifically, programs/applications can include computer readable program instructions that may be configured to carry out functions of embodiments of aspects described herein.

Examples of I/O devices 1408 include but are not limited to microphones, speakers, Global Positioning System (GPS) devices, RGB, IR, and/or spectral cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (1412) coupled to the computer system through one or more I/O interfaces 1410.

Computer system 1400 may communicate with one or more external devices 1412 via one or more I/O interfaces 1410. Example external devices include a keyboard, a pointing device, a display, and/or any other devices that enable a user to interact with computer system 1400. Other example external devices include any device that enables computer system 1400 to communicate with one or more other computing systems or peripheral devices such as a printer. A network interface/adapter is an example I/O interface that enables computer system 1400 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 1410 and external devices 1412 can occur across wired and/or wireless communications link(s) 1411, such as Ethernet-based wired or wireless connections. Example wireless connections include cellular, Wi-Fi, Bluetooth®, proximity-based, near-field, or other types of wireless connections. More generally, communications link(s) 1411 may be any appropriate wireless and/or wired communication link(s) for communicating data.

Particular external device(s) 1412 may include one or more data storage devices, which may store one or more programs, one or more computer readable program instructions, and/or data, etc. Computer system 1400 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Aspects of the present invention may be a system, a method, and/or a computer program product, any of which may be configured to perform or facilitate aspects described herein. Computer system configured to perform these and other methods, and computer program products that include a computer readable storage medium storing instructions for execution to perform these and other methods are also provided.

Computer system 1400 may be operational with numerous other general purpose or special purpose computing system environments or configurations. Computer system 1400 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C #, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

While several aspects of the present invention have been described and depicted herein, these are only examples, and alternative aspects may be affected by those skilled in the art to accomplish the same objectives. Accordingly, it is intended by the appended claims to cover all such alternative aspects as fall within the true spirit and scope of the invention.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

The following are non-limiting examples of aspects that may be claimed, and are provided to further illustrate the scope of the disclosure. In one example, a computer-implemented method includes identifying, as part of a surgical plan for a surgical procedure, one or more planned cut paths that an autonomous robotic surgical system is to follow in cutting patient anatomy using a cutting tool mounted to the autonomous robotic surgical system. The cutting tool includes a blade that moves in an oscillating or reciprocating motion to cut the patient anatomy. Each cut path of the one or more planned cut paths defines a respective path along which the autonomous robotic surgical system is to drive the blade in performing a respective cut. The method further includes building and providing an interface for display to a user, wherein the building includes in the interface one or more virtual representations of the patient anatomy and graphical representations of the identified one or more planned cut paths, including positioning and trajectory thereof relative to the patient anatomy.

In an example, the cutting tool comprises a saw, and the oscillating or reciprocating motion of the blade comprises an oscillatory back-and-forth cutting stroke in a plane in which the blade is aligned. In a further example, the saw comprises a sagittal saw.

In an example, each cut path of the one or more planned cut paths comprises a collection of ordered waypoints that the autonomous robotic surgical system is to follow during a cutting operation.

In an example, the one or more planned cut paths include a plurality of planned cut paths, and the building further includes providing, in the interface, visual indicators identifying a sequence in which two or more of the plurality of planned cut paths are to be performed relative to each other. In a further example, the visual indicators comprise at least one of differing colors, differing shadings, or differing opacities applied to respective cut paths of the plurality of planned cut paths.

In an example, the one or more planned cut paths include a plurality of planned cut paths, and the building further includes concurrently displaying in the interface a first graphical representation of at least one already-performed cut path having a first visual encoding, a second graphical representation of an in-progress cut path having a second visual encoding visually distinguishable from the first visual encoding, and a third graphical representation of at least one to-be-performed cut path having a third visual encoding visually distinguishable from each of the first visual encoding and the second visual encoding. In a further example, each of the first visual encoding, the second visual encoding, and the third visual encoding comprises a respective one or more of a color, a shading, or an opacity level that differs from the respective one or more of a color, a shading, or an opacity level of each other visual encoding.

In an example, the building further includes superimposing, on the interface, a graphical representation of a current position of the cutting tool on a cut path being executed by the autonomous robotic surgical system, and the graphical representation of the current position is updated in real time as the autonomous robotic surgical system drives the blade along the cut path. In a further example, the graphical representation of the current position comprises a graphical element representing the blade superimposed on the cut path being executed.

In an example, the building further includes displaying in the interface one or more virtual boundary constraints positioned relative to the one or more planned cut paths. Each virtual boundary constraint of the one or more virtual boundary constraints corresponds to a respective identified at-risk anatomic structure of the patient and is spatially positioned in the interface to convey a spatial relationship between the respective at-risk anatomic structure and the one or more planned cut paths. In a further example, the one or more virtual boundary constraints comprise gates that simulate lateral constraints of a virtual jig configured to constrict a trajectory and motion of the blade relative to the respective identified at-risk anatomic structure. In another example, the respective identified at-risk anatomic structure comprises at least one of a medial collateral ligament, a lateral collateral ligament, a posterior cruciate ligament, or a patellar tendon.

In an example, at least one cut path of the one or more planned cut paths comprises a non-linear trajectory adapted to at least one of a bone geometry of the patient anatomy or a proximity of a soft tissue structure of the patient. In a further example, the non-linear trajectory comprises a curved path along a bone boundary of the patient anatomy.

In an example, the building further includes displaying in the interface a graphical representation of a pre-operatively planned cut path and a graphical representation of an actually-executed cut path corresponding to the pre-operatively planned cut path, wherein the actually-executed cut path reflects one or more intraoperative deviations from the pre-operatively planned cut path. In a further example, the one or more intraoperative deviations are attributable to at least one of a bone density change, a soft tissue condition, or an anatomic deviation encountered during the surgical procedure. In another example, the graphical representation of the pre-operatively planned cut path and the graphical representation of the actually-executed cut path are concurrently displayed in the interface with visually distinguishable encodings.

In an example, the one or more planned cut paths comprise a plurality of distinct cut paths for performing a single planar resection of the patient anatomy, and the building further includes displaying in the interface graphical representations of each of the plurality of distinct cut paths and visual indicators identifying an execution order of the plurality of distinct cut paths for the single planar resection. In a further example, the single planar resection comprises one of a proximal tibial resection, a distal femur resection, a posterior femur resection, a posterior chamfer resection, an anterior chamfer resection, or an anterior femur resection. In another example, at least two of the plurality of distinct cut paths for the single planar resection have different trajectories relative to each other.

In an example, the surgical procedure comprises a total knee arthroplasty.

In an example, a computer system comprises a memory and a processing circuit in communication with the memory. The memory stores instructions that, when executed by the processing circuit, cause the computer system to identify, as part of a surgical plan for a surgical procedure, one or more planned cut paths that an autonomous robotic surgical system is to follow in cutting patient anatomy using a cutting tool mounted to the autonomous robotic surgical system. The cutting tool includes a blade that moves in an oscillating or reciprocating motion to cut the patient anatomy, and each cut path of the one or more planned cut paths defines a respective path along which the autonomous robotic surgical system is to drive the blade in performing a respective cut. The instructions further cause the computer system to build and provide an interface for display to a user, including in the interface one or more virtual representations of the patient anatomy and graphical representations of the identified one or more planned cut paths, including positioning and trajectory thereof relative to the patient anatomy.

In an example of the computer system, the cutting tool comprises a sagittal saw, and the oscillating or reciprocating motion of the blade comprises an oscillatory back-and-forth cutting stroke in a plane in which the blade is aligned.

In an example of the computer system, the instructions further cause the computer system to concurrently display in the interface a first graphical representation of at least one already-performed cut path having a first visual encoding, a second graphical representation of an in-progress cut path having a second visual encoding visually distinguishable from the first visual encoding, and a third graphical representation of at least one to-be-performed cut path having a third visual encoding visually distinguishable from each of the first visual encoding and the second visual encoding.

In an example of the computer system, the instructions further cause the computer system to superimpose, on the interface, a graphical representation of a current position of the cutting tool on a cut path being executed by the autonomous robotic surgical system, and to update the graphical representation of the current position in real time as the autonomous robotic surgical system drives the blade along the cut path.

In an example of the computer system, the instructions further cause the computer system to display in the interface one or more virtual boundary constraints. Each virtual boundary constraint corresponds to a respective identified at-risk anatomic structure of the patient and is spatially positioned in the interface to convey a spatial relationship between the respective at-risk anatomic structure and the one or more planned cut paths. In a further example, the respective identified at-risk anatomic structure comprises at least one of a medial collateral ligament, a lateral collateral ligament, a posterior cruciate ligament, or a patellar tendon.

In an example of the computer system, at least one cut path of the one or more planned cut paths comprises a non-linear trajectory adapted to at least one of a bone geometry of the patient anatomy or a proximity of a soft tissue structure of the patient.

In an example of the computer system, the instructions further cause the computer system to display in the interface a graphical representation of a pre-operatively planned cut path and a graphical representation of an actually-executed cut path corresponding to the pre-operatively planned cut path, wherein the actually-executed cut path reflects one or more intraoperative deviations from the pre-operatively planned cut path.

In an example of the computer system, the one or more planned cut paths comprise a plurality of distinct cut paths for performing a single planar resection of the patient anatomy, and the instructions further cause the computer system to display in the interface graphical representations of each of the plurality of distinct cut paths and visual indicators identifying an execution order of the plurality of distinct cut paths for the single planar resection.

In an example of the computer system, the computer system is in communication with the autonomous robotic surgical system.

In an example, a computer program product comprises a computer readable storage medium readable by a processing circuit. The computer readable storage medium stores instructions that, when executed by the processing circuit, cause the processing circuit to identify, as part of a surgical plan for a surgical procedure, one or more planned cut paths that an autonomous robotic surgical system is to follow in cutting patient anatomy using a cutting tool mounted to the autonomous robotic surgical system. The cutting tool includes a blade that moves in an oscillating or reciprocating motion to cut the patient anatomy, and each cut path of the one or more planned cut paths defines a respective path along which the autonomous robotic surgical system is to drive the blade in performing a respective cut. The instructions further cause the processing circuit to build and provide an interface for display to a user, including in the interface one or more virtual representations of the patient anatomy and graphical representations of the identified one or more planned cut paths, including positioning and trajectory thereof relative to the patient anatomy.

In an example of the computer program product, the instructions further cause the processing circuit to concurrently display in the interface a first graphical representation of at least one already-performed cut path having a first visual encoding, a second graphical representation of an in-progress cut path having a second visual encoding visually distinguishable from the first visual encoding, and a third graphical representation of at least one to-be-performed cut path having a third visual encoding visually distinguishable from each of the first visual encoding and the second visual encoding.

In an example of the computer program product, the instructions further cause the processing circuit to superimpose, on the interface, a graphical representation of a current position of the cutting tool on a cut path being executed by the autonomous robotic surgical system, and to update the graphical representation of the current position in real time as the autonomous robotic surgical system drives the blade along the cut path.

In an example of the computer program product, the instructions further cause the processing circuit to display in the interface one or more virtual boundary constraints. Each virtual boundary constraint corresponds to a respective identified at-risk anatomic structure of the patient and is spatially positioned in the interface to convey a spatial relationship between the respective at-risk anatomic structure and the one or more planned cut paths.

In an example of the computer program product, at least one cut path of the one or more planned cut paths comprises a non-linear trajectory adapted to at least one of a bone geometry of the patient anatomy or a proximity of a soft tissue structure of the patient.

In an example of the computer program product, the instructions further cause the processing circuit to display in the interface a graphical representation of a pre-operatively planned cut path and a graphical representation of an actually-executed cut path corresponding to the pre-operatively planned cut path, wherein the actually-executed cut path reflects one or more intraoperative deviations from the pre-operatively planned cut path.

In an example of the computer program product, the one or more planned cut paths comprise a plurality of distinct cut paths for performing a single planar resection of the patient anatomy, and the instructions further cause the processing circuit to display in the interface graphical representations of each of the plurality of distinct cut paths and visual indicators identifying an execution order of the plurality of distinct cut paths for the single planar resection.

What is claimed is:

1. A computer-implemented method comprising:
identifying, as part of a surgical plan for a surgical procedure, one or more planned cut paths that an autonomous robotic surgical system is to follow in cutting patient anatomy using a cutting tool mounted to the autonomous robotic surgical system, and wherein each cut path of the one or more planned cut paths defines a respective path along which the autonomous robotic surgical system is to drive the cutting tool in performing a respective cut; and
building and providing an interface for display to a user, wherein the building includes in the interface:
one or more virtual representations of the patient anatomy; and
graphical representations of the identified one or more planned cut paths, including positioning and trajectory thereof relative to the patient anatomy.

2. The method of claim 1, wherein the cutting tool comprises a sagittal saw, and wherein an oscillating or reciprocating motion of a blade of the sagittal saw comprises an oscillatory back-and-forth cutting stroke in a plane in which the blade is aligned.

3. The method of claim 1, wherein each cut path of the one or more planned cut paths comprises a collection of ordered waypoints that the autonomous robotic surgical system is to follow during a cutting operation.

4. The method of claim 1, wherein the one or more planned cut paths include a plurality of planned cut paths, and wherein the building further includes concurrently displaying in the interface:
a first graphical representation of at least one already-performed cut path having a first visual encoding;
a second graphical representation of an in-progress cut path having a second visual encoding visually distinguishable from the first visual encoding; and
a third graphical representation of at least one to-be-performed cut path having a third visual encoding visually distinguishable from each of the first visual encoding and the second visual encoding.

5. The method of claim 4, wherein each of the first visual encoding, the second visual encoding, and the third visual encoding comprises a respective one or more of a color, a shading, or an opacity level that differs from the respective one or more of a color, a shading, or an opacity level of each other visual encoding.

6. The method of claim 1, wherein the building further includes superimposing, on the interface, a graphical representation of a current position of the cutting tool on a cut path being executed by the autonomous robotic surgical system, wherein the graphical representation of the current position comprises a graphical element representing the cutting tool and is updated in real time as the autonomous robotic surgical system drives the cutting tool along the cut path.

7. The method of claim 1, wherein the building further includes displaying in the interface one or more virtual boundary constraints positioned relative to the one or more planned cut paths, wherein each virtual boundary constraint of the one or more virtual boundary constraints corresponds to a respective identified at-risk anatomic structure of the patient and is spatially positioned in the interface to convey a spatial relationship between the respective at-risk anatomic structure and the one or more planned cut paths.

8. The method of claim 7, wherein the one or more virtual boundary constraints comprise gates that simulate lateral constraints of a virtual jig configured to constrict a trajectory and motion of the cutting tool relative to the respective identified at-risk anatomic structure, and wherein the respective identified at-risk anatomic structure comprises at least one of a medial collateral ligament, a lateral collateral ligament, a posterior cruciate ligament, or a patellar tendon.

9. The method of claim 1, wherein at least one cut path of the one or more planned cut paths comprises a non-linear trajectory comprising a curved path along a bone boundary of the patient anatomy, wherein the non-linear trajectory is adapted to at least one of a bone geometry of the patient anatomy or a proximity of a soft tissue structure of the patient.

10. The method of claim 1, wherein the building further includes displaying in the interface:
a graphical representation of a pre-operatively planned cut path; and
a graphical representation of an actually-executed cut path corresponding to the pre-operatively planned cut path, wherein the actually-executed cut path reflects one or more intraoperative deviations from the pre-operatively planned cut path, and
wherein the graphical representation of the pre-operatively planned cut path and the graphical representation of the actually-executed cut path are concurrently displayed with visually distinguishable encodings.

11. The method of claim 10, wherein the one or more intraoperative deviations are attributable to at least one of a bone density change, a soft tissue condition, or an anatomic deviation encountered during the surgical procedure.

12. The method of claim 1, wherein the one or more planned cut paths comprise a plurality of distinct cut paths for performing a single planar resection of the patient anatomy, and
wherein the building further includes displaying in the interface graphical representations of each of the plurality of distinct cut paths and visual indicators identifying an execution order of the plurality of distinct cut paths for the single planar resection.

13. The method of claim 12, wherein at least two of the plurality of distinct cut paths for the single planar resection have different trajectories relative to each other.

14. The method of claim 12, wherein the single planar resection comprises one of a proximal tibial resection, a distal femur resection, a posterior femur resection, a posterior chamfer resection, an anterior chamfer resection, or an anterior femur resection of a total knee arthroplasty.

15. A computer system comprising:
a memory; and
a processing circuit in communication with the memory, wherein the memory stores instructions that, when executed by the processing circuit, cause the computer system to:
identify, as part of a surgical plan for a surgical procedure, one or more planned cut paths that an autonomous robotic surgical system is to follow in cutting patient anatomy using a cutting tool mounted to the autonomous robotic surgical system, wherein the cutting tool includes a blade that moves in an oscillating or reciprocating motion to cut the patient anatomy, and wherein each cut path of the one or more planned cut paths defines a respective path along which the autonomous robotic surgical system is to drive the blade in performing a respective cut; and
build and provide an interface for display to a user, including in the interface:
one or more virtual representations of the patient anatomy; and
graphical representations of the identified one or more planned cut paths, including positioning and trajectory thereof relative to the patient anatomy.

16. The computer system of claim 15, wherein the instructions further cause the computer system to concurrently display in the interface a first graphical representation of at least one already-performed cut path having a first visual encoding, a second graphical representation of an in-progress cut path having a second visual encoding visually distinguishable from the first visual encoding, and a third graphical representation of at least one to-be-performed cut path having a third visual encoding visually distinguishable from each of the first visual encoding and the second visual encoding.

17. The computer system of claim 15, wherein the instructions further cause the computer system to superimpose, on the interface, a graphical representation of a current position of the cutting tool on a cut path being executed by the autonomous robotic surgical system, and to update the graphical representation of the current position in real time as the autonomous robotic surgical system drives the blade along the cut path.

18. The computer system of claim 15, wherein the instructions further cause the computer system to display in the interface one or more virtual boundary constraints, wherein each virtual boundary constraint corresponds to a respective identified at-risk anatomic structure of the patient and is spatially positioned in the interface to convey a spatial relationship between the respective at-risk anatomic structure and the one or more planned cut paths.

19. A non-transitory computer program product comprising:
a computer readable storage medium readable by a processing circuit, the computer readable storage medium storing instructions that, when executed by the processing circuit, cause the processing circuit to:
identify, as part of a surgical plan for a surgical procedure, one or more planned cut paths that an autonomous robotic surgical system is to follow in cutting patient anatomy using a cutting tool mounted to the autonomous robotic surgical system, and wherein each cut path of the one or more planned cut paths defines a respective path along which the autonomous robotic surgical system is to drive the cutting tool in performing a respective cut; and
build and provide an interface for display to a user, including in the interface:
one or more virtual representations of the patient anatomy; and
graphical representations of the identified one or more planned cut paths, including positioning and trajectory thereof relative to the patient anatomy;
wherein the one or more planned cut paths comprise a plurality of distinct cut paths for performing a single planar resection of the patient anatomy, and wherein the instructions further cause the processing circuit to display in the interface graphical representations of each of the plurality of distinct cut paths and visual indicators identifying an execution order of the plurality of distinct cut paths for the single planar resection.

20. The non-transitory computer program product of claim 19, wherein the instructions further cause the processing circuit to display in the interface a graphical representation of a pre-operatively planned cut path and a graphical representation of an actually-executed cut path corresponding to the pre-operatively planned cut path, wherein the actually-executed cut path reflects one or more intraoperative deviations from the pre-operatively planned cut path.

21. The non-transitory computer program product of claim 19, wherein the cutting tool includes a blade that moves in an oscillating or reciprocating motion to cut the patient anatomy, and the interface includes a graphical representation of the blade in reference to at least one of the one or more planned cut paths.
